(19) United States
(12) Patent Application Publication
(10) Pub. No.: US 2018/0138834 A1
(43) Pub. Date: May 17, 2018

(54) ULTRASONIC ACTUATOR

(71) Applicant: SHARP KABUSHIKI KAISHA, Sakai City, Osaka (JP)

(72) Inventors: MASAKI HAMAMOTO, Sakai City (JP); HIDEKI ETOH, Sakai City (JP)

(21) Appl. No.: 15/574,974

(22) PCT Filed: Apr. 7, 2016
(86) PCT No.: PCT/JP2016/061422

§ 371 Date: Nov. 17, 2017

(30) Foreign Application Priority Data
May 21, 2015 (JP) 2015-103916

(51) Int. Cl. H02N 2/10; A61B 1/05; H02N 2/12; A61B 1/00

(52) U.S. Cl. CPC H02N 2/103; A61B 1/00147; H02N 2/126; A61B 1/05

(57) ABSTRACT

Provided is an ultrasonic actuator on which tuning that has little effect on vibration characteristics of a vibrator is performed. The ultrasonic actuator includes an ultrasonic vibrator (12), a housing (16) that rotates with vibration of the ultrasonic vibrator, and pantograph preload mechanisms (150, 151) that hold the ultrasonic vibrator at a node of vibration of the ultrasonic vibrator and generate pressure with which the ultrasonic vibrator is pressed against the housing.

### TECHNICAL FIELD

The present invention relates to an actuator that rotates a subject to be rotated, and particularly to an actuator that rotates, by utilizing ultrasonic vibration, a subject to be rotated.

### BACKGROUND ART

With development of robots in small sizes and precision apparatuses, actuators that are small and have high power-to-weight ratios have been demanded. Among them, an ultrasonic motor using a piezoelectric element such as a piezo element has a high power-to-weight ratio and is thus put into practical use, for example, for driving a camera lens. In particular, various configurations in which an ultrasonic vibrator is accommodated in a rotor have been proposed because such configuration is suitable for reduction in a size of an apparatus.

Meanwhile, the ultrasonic motor has amplitude expanded by using a resonance phenomenon, and is thus required to be subjected to tuning during manufacturing or after manufacturing in order to deal with, for example, an error in shape and lack of uniformity of materials during manufacturing. For dealing with such a situation, in an invention of PTL 1, for example, a compression spring that pushes out a contact section of a vibrating body functioning as a stator is provided in a main body of the vibrating body and the contact section and the compression spring are solidified with adhesive, and thus tuning of an ultrasonic motor is realized.

### CITATION LIST

### Patent Literature

PTL 1: Japanese Unexamined Patent Application Publication No. 2009-153283 (published on Jul. 9, 2009)

### SUMMARY OF INVENTION

### Technical Problem

In the ultrasonic motor of PTL 1, however, a preload adjustment mechanism such as the compression spring is provided in the main body of the vibrating body, which causes a problem that reaction force from a rotor affects a vibration mode of a vibrator. In particular, when a mass of the vibrating body varies due to individual difference in a volume of adhesive injected, a resonance mode of the vibrating body changes. That is, there is a concern that the tuning for optimizing vibration of the vibrator may, on the contrary, deteriorate vibration performance.

The invention was made in view of the aforementioned problems, and an object thereof is to provide an ultrasonic actuator on which tuning that has little effect on vibration characteristics of a vibrator is able to be performed.

### Solution to Problem

In order to solve the aforementioned problems, an ultrasonic actuator according to an aspect of the invention includes an ultrasonic vibrator, a rotor that rotates with vibration of the ultrasonic vibrator, and a preload mechanism that holds the ultrasonic vibrator at a node of vibration of the ultrasonic vibrator and generates pressure with which the ultrasonic vibrator is pressed against the rotor.

### Advantageous Effects of Invention

According to an aspect of the invention, it is possible to provide an ultrasonic actuator on which tuning that has little effect on vibration characteristics of an ultrasonic vibrator is able to be performed.

### BRIEF DESCRIPTION OF DRAWINGS

FIG. 1 is a schematic view illustrating a use form of a medical apparatus according to an embodiment of the invention.

FIG. 2 illustrates an external appearance of an insertion-section conveying unit according to the embodiment of the invention.

FIG. 3 is a schematic view illustrating a schematic configuration of the insertion-section conveying unit according to the embodiment of the invention.

FIG. 4 is a schematic view illustrating a form of an operation of the insertion-section conveying unit according to the embodiment of the invention.

FIG. 5 is a schematic view illustrating another form of the operation of the insertion-section conveying unit according to the embodiment of the invention.

FIG. 6 is a schematic view illustrating a schematic configuration of an in-wheel motor usable for the insertion-section conveying unit according to the embodiment of the invention.

FIG. 7 is a schematic view illustrating a schematic configuration of an ultrasonic vibrator usable for the in-wheel motor usable for the insertion-section conveying unit according to the embodiment of the invention.

FIG. 8 is a schematic view illustrating a vibration mode of the ultrasonic vibrator according to the embodiment of the invention.

FIG. 9 is a schematic view illustrating another vibration mode of the ultrasonic vibrator according to the embodiment of the invention.

FIG. 10 illustrates schematic views (a) and (b) both illustrating a conveyance principle of a rotor of the ultrasonic vibrator according to the embodiment of the invention.

FIG. 11 is a schematic view illustrating an overview of a preload holding mechanism by which the ultrasonic vibrator according to the embodiment of the invention is pressed against the rotor, in which (a) illustrates a state before an adjustment screw is tightened and (b) illustrates a state after the adjustment screw is tightened.

FIG. 12 is a schematic view illustrating a schematic configuration of an insertion-section conveying unit usable for a medical apparatus according to a second embodiment of the invention.

FIG. 13 illustrates positions of conveyance rollers on guides in the insertion-section conveying unit usable for the medical apparatus according to the second embodiment of the invention.

FIG. 14 illustrates views (a) and (b) both indicating a resultant vector of frictional force by conveyance rollers.

FIG. 15 is a schematic view illustrating a schematic configuration of an insertion-section conveying unit usable for a medical apparatus according to a third embodiment of the invention.

FIG. 16 illustrates views (a) and (b) both indicating a resultant vector of frictional force when conveyance rollers rotate as illustrated in FIG. 5.

FIG. 17 is a schematic view illustrating a schematic configuration of an insertion-section conveying unit usable for a medical apparatus according to a fourth embodiment of the invention.

FIG. 18 is a schematic view illustrating a schematic configuration of an insertion-section conveying unit usable for a medical apparatus according to a fifth embodiment of the invention.

### DESCRIPTION OF EMBODIMENTS

### Embodiment 1

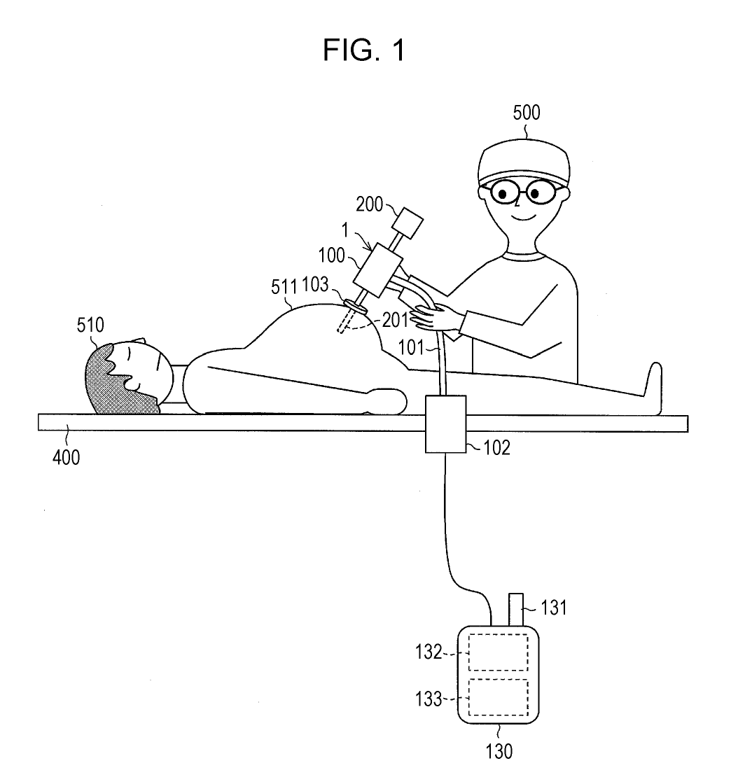

An embodiment of the invention will be described in detail below with reference to FIGS. 1 to 11.

### (Overview of Medical Apparatus 1)

FIG. 1 is a schematic view illustrating an example of a use form of a medical apparatus 1 according to the embodiment of the invention. The medical apparatus 1 is an apparatus that adjusts a position of a rigid endoscope 200. In the present embodiment, as an example to which the invention is applied, it is assumed that an insertion section (sheath tube) 201 of the rigid endoscope 200 is inserted into a body cavity of an abdomen 511 of a patient 510 lying on a surgery table 400, and a surgeon 500 performs a surgery on the basis of an image obtained from an image sensor positioned at a distal end of the insertion section 201.

In FIG. 1, the medical apparatus 1 includes an insertion-section conveying unit 100, a flexible arm (actuator securing unit) 101, a stand (actuator securing unit) 102, a surgical port 103, a control unit (control device) 130, and the rigid endoscope 200. Note that, the insertion-section conveying unit 100 and the control unit 130 will be described in detail later.

The flexible arm 101 supports and secures the insertion-section conveying unit 100 at one end thereof and can be manually bent into a desired shape. Specifically, the flexible arm 101 sets and secures the insertion-section conveying unit 100 at a position desired by the surgeon 500.

The stand 102 secures the other end of the flexible arm 101 so as to secure the flexible arm 101 toward the patient 510 lying on the surgery table 400. The stand 102 is installed (fixed) at the surgery table 400.

The surgical port 103 is a medical instrument having a through-hole for inserting a medical instrument into the body cavity of the patient 510 and is disposed on a surface of the abdomen 511 of the patient 510. The surgical port 103 is not essential depending on a surgical method and is not an essential element of the present embodiment.

Although the rigid endoscope 200 having the insertion section 201 in a cylindrical (rod) shape is used as an example of a medical instrument in the present embodiment, the medical instrument is not limited thereto. A medical instrument having an insertion section in a rod (columnar) shape for inserting a medical instrument into the body of the patient 510 may be used in place of the rigid endoscope 200. For example, a medical instrument having a surgical instrument, such as a forceps, provided at the distal end of an insertion section in a columnar shape, a catheter in a columnar shape also functioning as an insertion section, or the like may be used as the medical instrument. Such medical instruments are generally referred to as operation elements in the invention.

### (Configuration of Insertion-Section Conveying Unit)

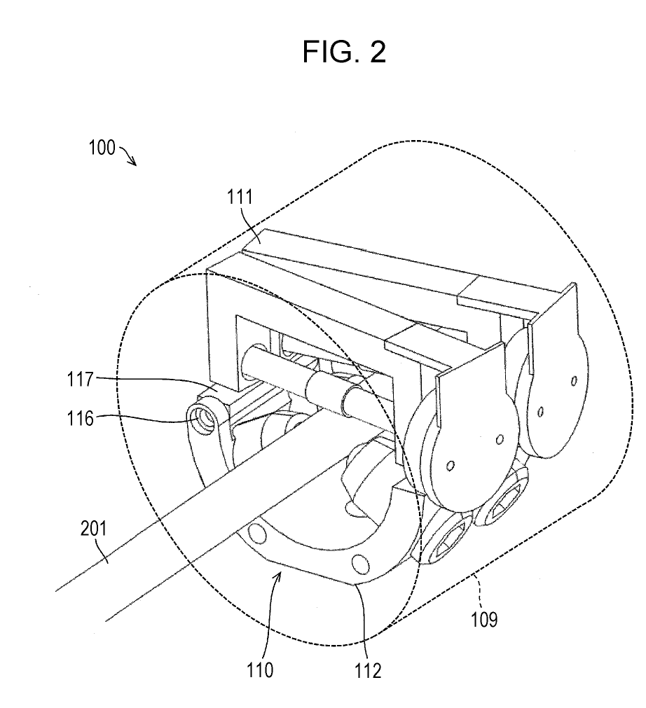

FIG. 2 is a perspective view illustrating a schematic configuration of the insertion-section conveying unit 100. As illustrated in FIG. 2, the insertion-section conveying unit 100 includes an actuator holder (actuator securing unit) 109 and a differential drive mechanism (actuator, friction drive actuator) 110. The differential drive mechanism 110 includes a plurality of conveyance rollers (a front conveyance roller 1112 and a rear conveyance roller 1113) that are able to convey the insertion section (operation element) 201 in a rod shape in a long axis direction thereof and rotate the insertion section 201 about the long axis.

The actuator holder 109 is a hollow housing that holds the differential drive mechanism 110. One end of the flexible arm 101 (refer to FIG. 1) is secured to a side surface of the actuator holder 109. The actuator holder 109, the flexible arm 101, and the stand 102 constitute an actuator securing unit for securing the differential drive mechanism 110 to an area near a surgical site.

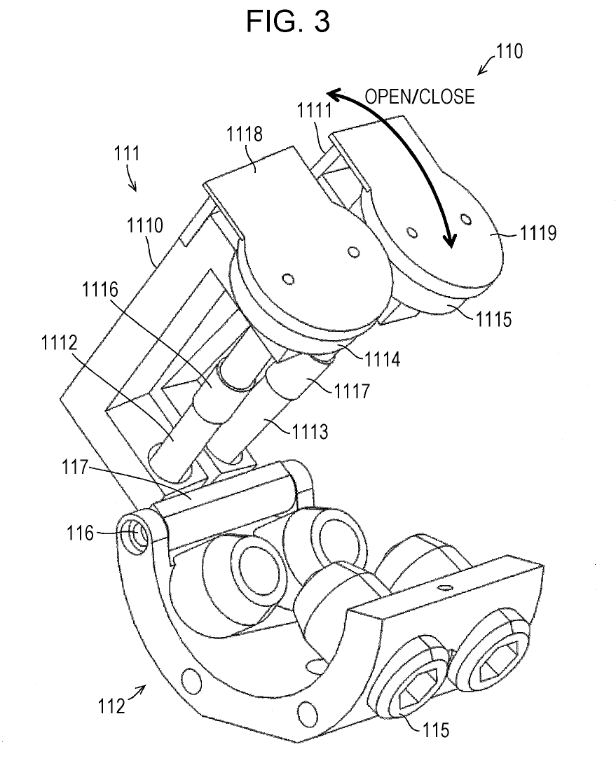

FIG. 3 is a perspective view illustrating a schematic configuration of the differential drive mechanism 110. As illustrated in FIG. 3, the differential drive mechanism 110 includes an upper housing unit 111, a lower housing unit 112, a coupling section 117, and a preload spring (restoring section) 116.

The upper housing unit 111 includes a front arm 1110 and a rear arm 1111. The front arm 1110 includes the front conveyance roller 1112, a front in-wheel motor (ultrasonic actuator, ultrasonic motor) 1114 that drives the front conveyance roller 1112, and a rubber roller 1116. The rear arm 1111 includes the rear conveyance roller 1113, a rear in-wheel motor (ultrasonic actuator, ultrasonic motor) 1115 that drives the rear conveyance roller 1113, and a rubber roller 1117.

The rubber rollers 1116 and 1117 are elastic frictional materials that are arranged on surfaces of the front conveyance roller 1112 and the rear conveyance roller 1113, respectively. Thus, frictional force between the insertion section 201 and each of the conveyance rollers increases, so that revolving of the conveyance rollers without contact is able to be prevented.

The rubber rollers 1116 and 1117 are arranged so as to be detachable from the front conveyance roller 1112 and the rear conveyance roller 1113, respectively. Thus, the robber rollers 1116 and 1117 are able to be easily replaced in a case of being damaged or contaminated.

Specifically, each of the rubber rollers 1116 and 1117 may have a groove extending substantially parallel to the long axis of the insertion section 201. The rubber rollers 1116 and 1117 may be configured to be able to be pulled out while the front in-wheel motor 1114 and the rear in-wheel motor 1115 are being detached from the front conveyance roller 1112 and the rear conveyance roller 1113.

The lower housing unit 112 includes four ball bearings (holders, sliders) 115. The ball bearings 115 hold the insertion section 201 between the ball bearings 115 and the front conveyance roller 1112 and rear conveyance roller 1113.

The coupling section 117 is a member that has a function as a hinge coupling the upper housing unit 111 and the lower housing unit 112 in an openable-closable manner. A distance between the upper housing unit 111 and the lower housing unit 112 that are coupled by the coupling section 117 varies in accordance with a thickness of the insertion section 201. That is, the coupling section 117 changes distances between the front conveyance roller 1112 and rear conveyance roller 1113 and the ball bearings 115 in accordance with the thickness of the insertion section 201.

The preload spring 116 applies restoring force in a direction in which the upper housing unit 111 and the lower housing unit 112 are closed together. When closed together, the upper housing unit 111 and the lower housing unit 112 form a ring-shaped housing.

When the upper housing unit 111 and the lower housing unit 112 are closed together, the rubber roller 1116 in the front conveyance roller 1112, the rubber roller 1117 in the rear conveyance roller 1113, and the four ball bearings 115 are pressed against a side surface of the insertion section 201 by the restoring force of the preload spring 116. Hereinafter, unless otherwise noted, the rollers including the rubber roller sections are referred to as conveyance rollers for simplification of description.

The conveyance rollers are arranged for the respective arms so as to be rotatable via bearings that are not illustrated. The upper housing unit 111 and the lower housing unit 112 are joined to each other in an openable-closable manner. In a state where a position of the differential drive mechanism 110 relative to a surgical site within a body cavity is fixed by the actuator holder 109, the differential drive mechanism 110 conveys the insertion section 201 of the rigid endoscope 200 in a translation or rotation direction. The translation direction is a direction parallel to the long axis direction of the insertion section 201 and the rotation direction is a rotation direction about the long axis of the insertion section 201. Note that, the rigid endoscope 200 is constituted by a grip section and the insertion section 201 and the insertion section 201 has a cylindrical shape.

On surfaces of the front conveyance roller 1112 and the rear conveyance roller 1113, concave steps whose widths are equal to or greater than widths of the rubber rollers 1116 and 1117 are provided. Therefore, positions of the rubber rollers 1116 and 1117 are able to be prevented from being significantly shifted, because of friction with the insertion section 201.

According to the aforementioned configuration, the position of the insertion section 201 of the rigid endoscope 200 in a direction vertical to the axis direction of the insertion section 201 is restricted by the front conveyance roller 1112, the rear conveyance roller 1113, and the two ball bearings 115 (FIG. 2).

On the other hand, when the upper housing unit 111 and the lower housing unit 112 are opened, the front conveyance roller 1112 and the rear conveyance roller 1113 are separated from the ball bearings 115, so that the insertion section 201 is released from the differential drive mechanism 110. By doing so, it is possible to remove the rigid endoscope 200 from the insertion-section conveying unit 100 and the removed rigid endoscope 200 may be singly subjected to cleaning or the like.

Here, the ball bearings 115 come into point contact with the side surface of the insertion section 201. Therefore, though it is sufficient to use two ball bearings 115 in addition to the front conveyance roller 1112 and the rear conveyance roller 1113, the four ball bearings are used here in consideration of position adjustment for operation of the insertion section 201. Alternatively, five or more ball bearings may be used.

### (Driving Principle of Insertion Section 201)

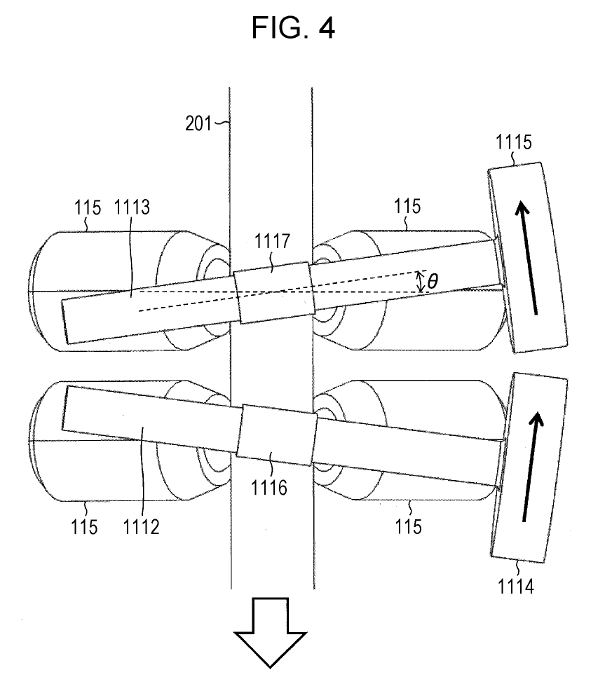

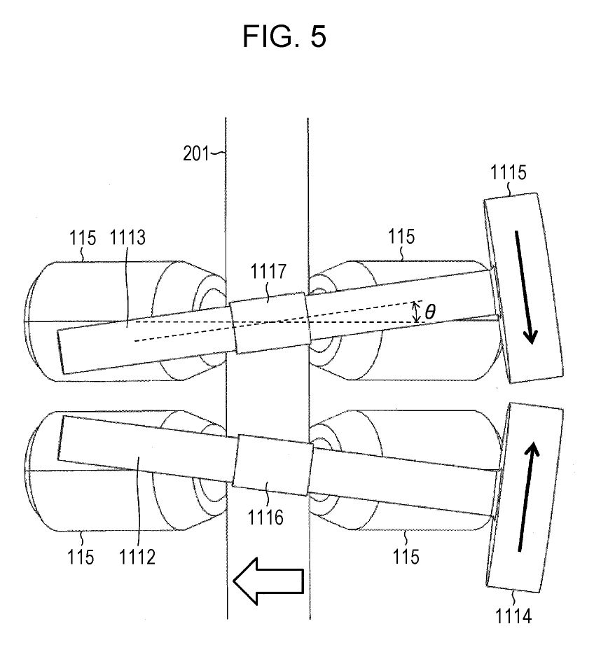

FIGS. 4 and 5 each illustrate a form of operation of the insertion-section conveying unit 100. In the present embodiment, translation and rotation operations of the insertion section 201 are realized by differential drive similarly to the invention of PTL 1. That is, when both the conveyance rollers rotate in the same direction as illustrated in FIG. 4, the insertion section 201 is conveyed in the translation direction with resultant force of frictional force applied to the insertion section 201. When the conveyance rollers rotate in reverse directions as illustrated in FIG. 5, the insertion section 201 is conveyed in the rotation direction with the resultant force of the frictional force applied to the insertion section 201.

At this time, when ϕ is a diameter of the conveyance rollers, θ is a crossing angle, ω is a rotation speed of the conveyance rollers, and D is a diameter of the insertion section 201,

a delivery speed in the case of rotation in the same direction is expressed by

vₜᵣₐₙₛ=π*ϕ*ω*cos(θ), and

a rotation speed in the case of the rotation in the reverse directions is expressed by

vᵣₒₜ=ϕ*ω*sin(θ)/D.

The crossing angle θ is an angle between the rotation axis of each of the conveyance rollers and a line normal to the long axis of the insertion section 201.

Here, since the front arm 1110 and the rear arm 1111 are movable as illustrated in FIG. 3, the crossing angle ϕ is able to be changed, but this is not essential. In the present embodiment, the crossing angle ϕ is assumed to be fixed to a predetermined angle by adjustment before a surgery. Note that, a value itself of the angle is a matter of design choice depending on many factors such as an outer diameter, a mass, and a friction coefficient of a subject to be conveyed, and a conveyance speed that a surgeon prefers, and is not a matter that determines whether the invention can be applied.

### (In-Wheel Motor)

### (Entire Configuration)

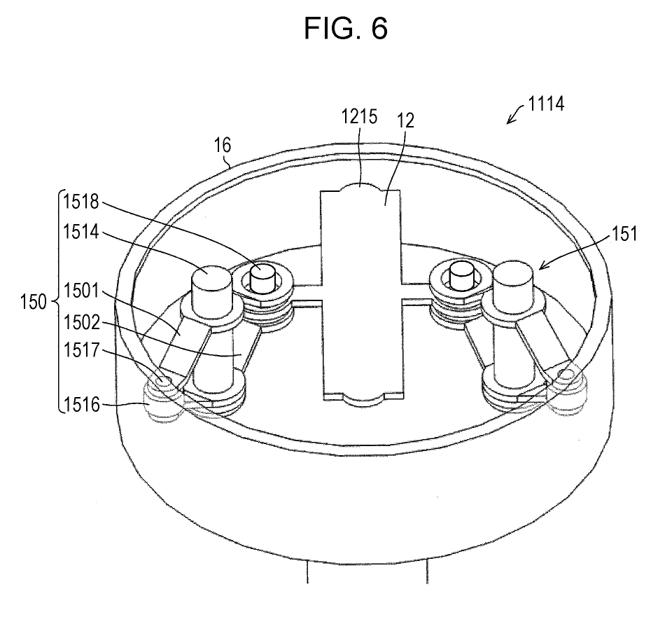

FIG. 6 illustrates a configuration of the front in-wheel motor 1114. Note that, a configuration of the rear in-wheel motor 1115 is similar to the configuration of the front in-wheel motor 1114, and thus is not described with reference to the drawings.

As illustrated in FIGS. 6 and 3, the front in-wheel motor 1114 includes an ultrasonic vibrator 12, pantograph preload mechanisms 150 and 151, a housing 16, and a motor cover 1118. The rear in-wheel motor 1115 includes a motor cover 1119 instead of the motor cover 1118.

Each of the front in-wheel motor 1114 and the read in-wheel motor 1115 is configured so that the ultrasonic vibrator 12 that has a function of conveying the housing (rotor) 16 by a distal end thereof being elliptically moved is pressed against the housing 16 by two pantograph preload mechanisms 150 and 151.

Each of the two pantograph preload mechanisms 150 and 151 holds the ultrasonic vibrator 12 at a node of vibration of the ultrasonic vibrator 12 and generates pressure for pressing the ultrasonic vibrator 12 against the housing 16. The pantograph preload mechanisms 150 and 151 are fixed to the motor cover 1118 (or the motor cover 1119) and the motor cover 1118 (or the motor cover 1119) is fixed to the front arm 1110 (or the rear arm 1111).

The housing 16 is configured to be held rotatably with respect to the front arm 1110 (or the rear arm 1111), and thus the housing 16 rotates with respect to the front arm 1110 and the rear arm 1111 by frictional force applied from the ultrasonic vibrator 12 to the housing 16.

### (Main Configuration of Ultrasonic Vibrator 12)

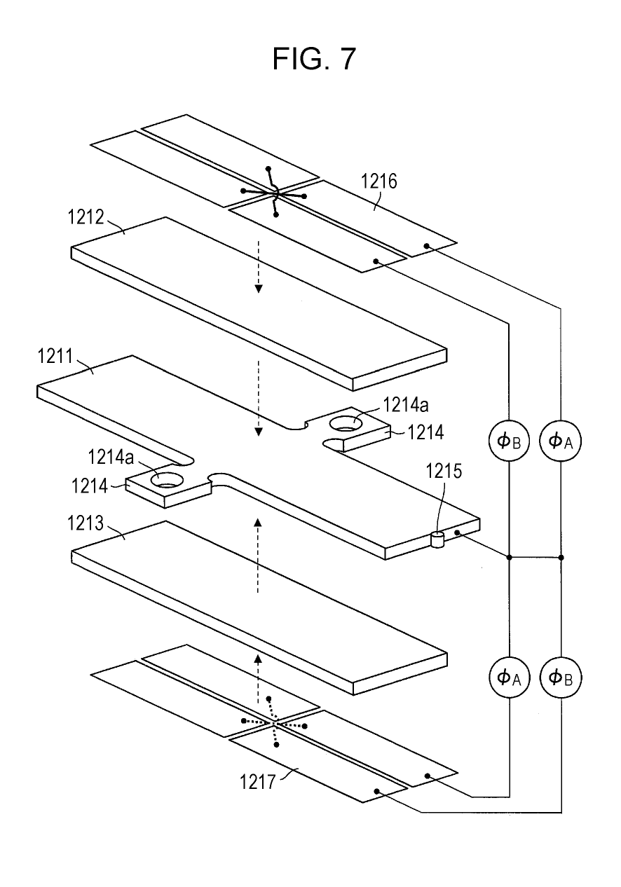

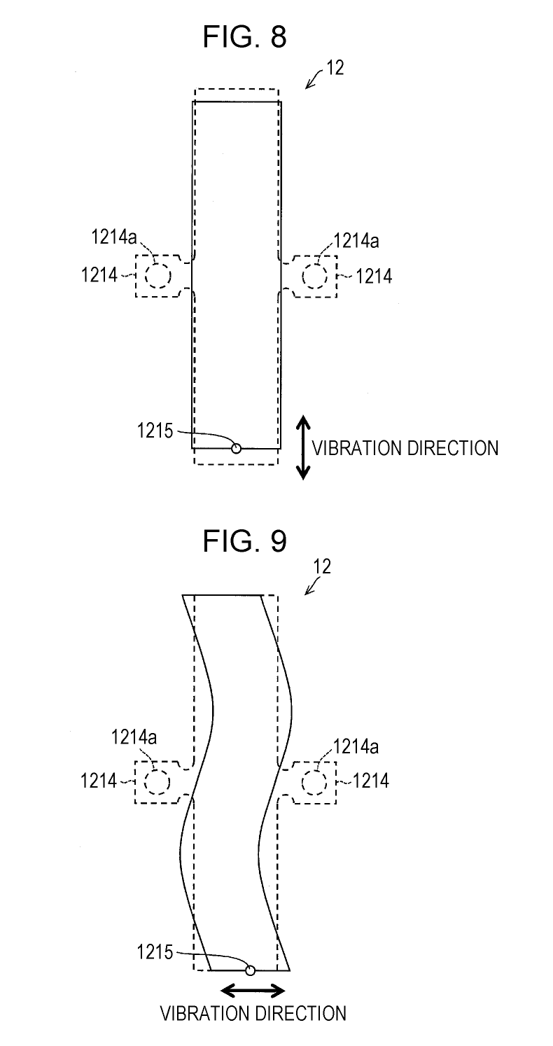

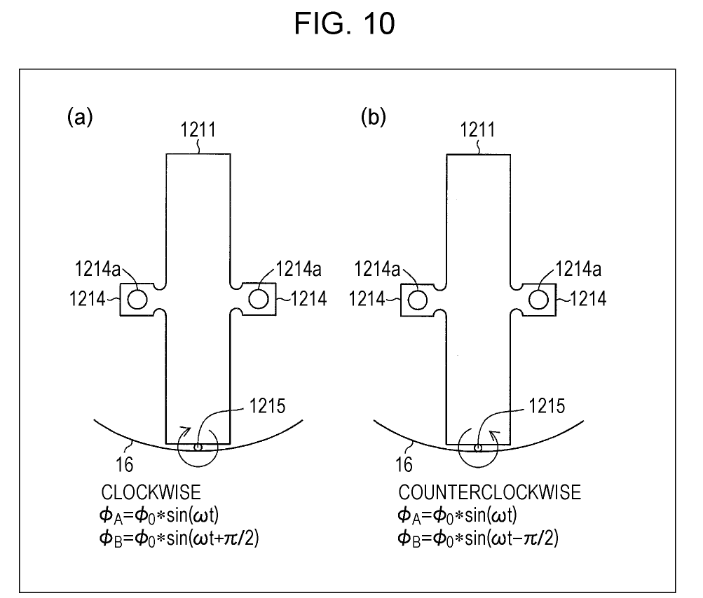

A representative configuration and function of the ultrasonic vibrator 12 usable in the insertion-section conveying unit 100 according to the present embodiment will be described with reference to FIGS. 7 to 10. FIG. 7 is a schematic view illustrating a schematic configuration of the ultrasonic vibrator 12. FIGS. 8 and 9 are schematic views each illustrating a vibration mode of the ultrasonic vibrator 12. FIG. 10 is a schematic view illustrating a principle of rotation of the housing (also serving as the rotor) 16 by the ultrasonic vibrator 12.

As illustrated in FIG. 7, the ultrasonic vibrator 12 includes a diaphragm 1211, an upper PZT (Lead Zirconate Titanate) element 1212, a lower PZT element 1213, an upper electrode 1216, and a lower electrode 1217.

The ultrasonic vibrator 12 has the upper PZT element 1212 in a rectangular shape and the lower PZT element 1213 in a rectangular shape placed on respective surfaces of the diaphragm 1211 in a substantially rectangular shape. The upper PZT element 1212 has the upper electrode 1216, which is divided into four pieces, placed on a surface opposite to the diaphragm 1211 and the lower PZT element 1213 has the lower electrode 1217, which is divided into four pieces in the same manner, placed on a surface opposite to the diaphragm 1211. Each of the upper PZT element 1212 and the lower PZT element 1213 is polarized in parallel to a direction directed to the diaphragm 1211 and is deformed by a piezoelectric effect with respect to an electric field in the direction.

A contact section (distal end) 1215 that contacts with the housing 16 is provided in one of short sides of the diaphragm 1211 in the substantially rectangular shape.

The ultrasonic vibrator 12 has holders 1214 each of which is a projection formed at a node of standing-wave vibration caused by exciting the ultrasonic vibrator 12. Specifically, the holders 1214 are provided respectively at the center of two long sides of the diaphragm 1211. Each holder 1214 has a hole 1214a.

In the present embodiment, a size of the rectangular portion of the diaphragm 1211 of the ultrasonic vibrator 12 is 9 mm in length and 2 mm in width, and size of the upper and lower PZT elements is 8 mm in length and 2 mm in width. All of them has a thickness of 0.2 mm. The diaphragm 1211 is made of stainless steel and each of the PZT elements is made of a material that is generally referred to as hard-type lead zirconate titanate (Pb(Ti.Zr)O₃). However, such configurations are merely examples of configurations used for an experiment by the inventors of the invention and do not narrow the scope of the invention. The invention is to be applied to an ultrasonic motor that receives a frictional force by preloading and rotates, and actuators in general.

### (Driving Principle)

The ultrasonic vibrator 12 has two types of vibration modes of a vertical-direction primary vibration mode (hereinafter, referred to as stretching vibration) and a deflection (bending) tertiary vibration mode (hereinafter, referred to as bending vibration). In the present embodiment, resonance frequencies of the stretching vibration and the bending vibration are both 240 kHz. Note that, such a numerical value is applied in the case of the aforementioned shapes and varies in accordance with a matter of design choice. Such a numerical value is not a matter that determines whether the invention can be applied.

The vibration excited in the two types of vibration modes described above is standing-wave vibration in which a position of the node does not vary. As described above, the holder 1214 is positioned at a location corresponding to the node of the standing-wave vibration caused by exciting the ultrasonic vibrator 12.

The stretching vibration is excited when the same voltage is applied to all the four divided electrodes, and the bending vibration is excited when the same voltage is applied to electrodes located diagonally and voltages having opposite polarities are applied to adjacent electrodes among the four divided electrodes.

In the present embodiment, the electrodes located diagonally are short-circuited and the electrodes adjacent to each other are insulated. Hereinafter, the voltages applied to the electrodes that are insulated are denoted by ϕ_{A} and ϕ_{B}. When alternating current of the same phase of 240 kHz is applied to ϕ_{A} and ϕ_{B}, the stretching vibration illustrated in FIG. 8 is excited, and in the case where alternating current of reverse phase is applied, the bending vibration illustrated in FIG. 9 is excited.

Thus, when the bending vibration is excited being shifting by ±90° relative to the stretching vibration, vibration in which the stretching vibration and the bending vibration are combined with the phase shifted by ±90° is excited. As a result, the contact section 1215 of the ultrasonic vibrator is elliptically moved as illustrated in (a) and (b) of FIG. 10.

Note that, though a method for driving the rectangular vibrator having four divided electrodes is described here for convenience of the description, a driving method is not limited thereto, because a main feature of the invention is to adopt the friction drive motor for driving in an internal contact manner. For example, ϕ_{A} and ϕ_{B} are set as sine waves, but are not limited thereto and may be square waves or sawtooth waves. Though the phase shift is set as ±90° for convenience of waveform generation, the phase shift is not limited thereto because conveyance is enabled substantially as long as the elliptical movement described above is caused. Further, there is also a method for enabling conveyance in two directions even in the case of a single phase, for example, by means of different vibration modes being established in accordance with a driving frequency. Derivatives thereof are also able to be applied to the invention.

### (Motor Cover)

The motor covers 1118 and 1119 are bases for supporting the pantograph preload mechanisms 150 and 151 and have a function of protecting the ultrasonic vibrator 12 against contaminants such as blood.

Each of the motor covers 1118 and 1119 has an adjustment hole (not illustrated) into which an adjustment screw 1514 for adjusting stretching of each of the pantograph preload mechanisms 150 and 151 is inserted.

### (Housing 16)

The housing 16 functions as the rotor by itself and has a function of protecting the ultrasonic vibrator 12 against contaminants such as blood.

The housing 16 is desired to be made of a material having an abrasion resistance because the housing 16 receives frictional force from the ultrasonic vibrator 12. According to an investigation by the inventors of the invention, it is effective to adopt, for example, steel subjected to high frequency hardening or dry carbon.

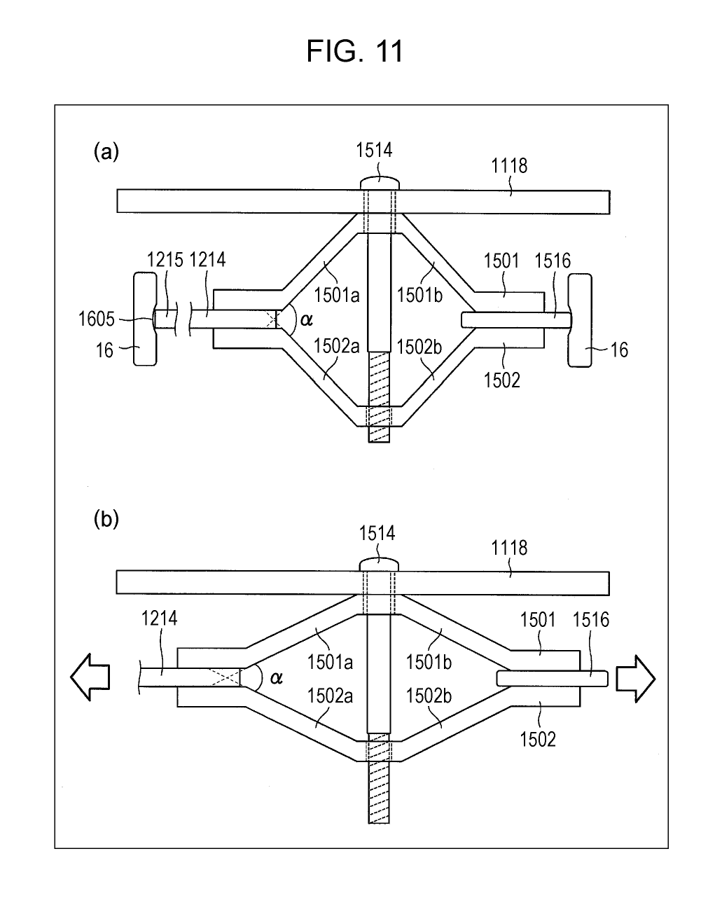

The housing 16 has a guide groove 1605 (refer to FIG. 11) that restricts a position where the contact section 1215 of the ultrasonic vibrator 12 contacts with the housing 16. Thus, the ultrasonic vibrator 12 is able to rotate the housing 16 stably.

### (Pantograph Preload Mechanisms 150 and 151)

(a) and (b) of FIG. 11 are schematic views each illustrating an overview of the pantograph preload mechanism 150. As illustrated in the views, the pantograph preload mechanism 150 includes metal fittings 1501 and 1502 each of which has a substantially V-shape, the adjustment screw (adjustment member) 1514, and a guide roller (slide support section) 1516.

The metal fittings 1501 and 1502 respectively have arms 1501a and 1501b and arms 1502a and 1502b. The arm 1501a and the arm 1502a constitute a pair and the arm 1501b and the arm 1502b also constitute a pair. In this manner, each of the pantograph preload mechanisms 150 and 151 includes two pairs of arms. One end of each of the pair of the arm 1501a and the arm 1502a is connected to the ultrasonic vibrator 12 with an angle formed, and the pantograph preload mechanism 150 is configured to adjust pressure, with which the ultrasonic vibrator 12 is pressed against the housing 16, by adjusting an angle α formed between the arm 1501a and the arm 1502a at the end.

Each end of the arm 1501b and the arm 1502b is connected to the guide roller 1516 by a guide pin 1517 (refer to FIG. 6).

A configuration of the pantograph preload mechanism 151 is similar to that of the pantograph preload mechanism 150. Note that, the pantograph preload mechanisms 150 and 151 may be single-arm pantographs including one pair of arms.

The present embodiment adopts a mechanism in which by tightening up the face-to-face metal fittings 1501 and 1502 by the adjustment screw 1514, a pantograph of the pantograph preload mechanisms 150 and 151 is stretched. As illustrated in FIG. 11, the adjustment screw 1514 is inserted into the adjustment hole formed in each of the motor covers 1118 and 1119 and a head of the adjustment screw 1514 (a part of the adjustment screw 1514) is exposed to an outer surface of the motor covers 1118 and 1119.

The pantograph preload mechanisms 150 and 151 are connected to the holders 1214 formed at the node of vibration of the ultrasonic vibrator 12.

Specifically, the holders 1214 are provided with the holes 1214a (refer to FIG. 7). The holes 1214a and holes (not illustrated) provided in one ends of the pantograph preload mechanisms 150 and 151 are connected by guide pins 1518 (refer to FIG. 6). The guide pins 1518 are pins by which the ultrasonic vibrator 12 is connected to the holders 1214.

As described above, the holders 1214 are provided respectively at the center of the two long sides of the diaphragm 1211. That is, the holders 1214 are formed to be positioned symmetrically about a long axis of the ultrasonic vibrator 12, and the pantograph preload mechanisms 150 and 151 are connected to the respective holders 1214 that constitute a pair.

Such configurations make it possible for the pantograph preload mechanisms 150 and 151 to stably hold the ultrasonic vibrator 12.

As described above, the one end of the pantograph preload mechanism 150 is connected to the hole of the holder 1214 of the ultrasonic vibrator 12 by the guide pin 1518. The other end of the pantograph preload mechanism 150 is provided with the guide roller 1516 contacting with an inner surface of the housing 16, by which the housing 16 is smoothly held.

In this manner, the pantograph preload mechanisms 150 and 151 have one ends at which the holders 1214 of the ultrasonic vibrator 12 are held and the other ends at which the housing 16 is held by the guide rollers. The housing 16 is held from inside at three portions in total of two portions of the guide rollers and the contact section 1215 of the ultrasonic vibrator 12 and rotates.

At this time, as described above, the holders 1214 are at positions corresponding to nodes of standing-wave vibration caused by exciting the ultrasonic vibrator 12. Thus, the pantograph preload mechanisms 150 and 151 are able to hold the ultrasonic vibrator 12 without interfering with vibration of the ultrasonic vibrator 12.

The metal fitting 1501 is bonded to the motor cover 1118, for example, by adhesive or a screw and a screw hole which are not illustrated. Each of the motor cover 1118 and the metal fitting 1501 is provided with a through hole and the metal fitting 1502 is provided with a screw hole, so that a threaded part of the adjustment screw 1514 is engaged only with the metal fitting 1502 and semi-fixed.

As indicated with a change from (a) to (b) of FIG. 11, by tightening the adjustment screw 1514, the pantograph preload mechanism 150 is deformed so as to be laterally extended. That is, the pantograph preload mechanism 150 is deformed so as to separate the ultrasonic vibrator 12 and the guide roller 1516 from each other. Actually, as a distance of the separation is almost fixed, when the adjustment screw 1514 is tightened up, the contact section 1215 of the ultrasonic vibrator 12 is pressed against the housing 16 through elastic deformation of the metal fittings 1501 and 1502.

Specifically, the pantograph preload mechanisms 150 and 151 generate force in a direction in which a distance between the node (holder 1214) of the vibration of the ultrasonic vibrator 12 and the guide roller 1516 increases, thereby adjusting pressure with which the ultrasonic vibrator 12 is pressed against the housing 16.

When the distance between the node of the vibration of the ultrasonic vibrator 12 and the guide roller 1516 is differentiated between the pantograph preload mechanism 150 and the pantograph preload mechanism 151, it is also possible to adjust a contact angle at which the contact section 1215 is pressed against the housing 16.

Though shapes of the pantograph preload mechanisms 150 and 151 are not limited as long as they do not depart from the purpose of preloading adjustment, it is desirable that simplicity of manufacture and adjustment is considered.

### (Configuration of Control Unit 130)

As illustrated in FIG. 1, the control unit 130 includes a command input section 131, a drive signal generator (voltage supplier, operation command section) 132, and a battery 133 that supplies electric power to these components. The control unit 130 is detachably connected to the insertion-section conveying unit 100 by a cable extending via the stand 102 and the flexible arm 101.

The command input section 131 is an input device used by an operator (user) for inputting a command and is, for example, an input device such as a joystick. For example, the operator manually tilts the joystick forward, rearward, leftward, or rightward so as to input a command for conveying (translating or rotating) the insertion section 201 of the rigid endoscope 200. The command input section 131 outputs the command input by the operator to the drive signal generator 132. The command input by the operator designates, for example, a moving direction and a moving speed of the insertion section 201.

On the basis of the command input by the operator, the drive signal generator 132 generates drive signals for exciting the upper PZT element 1212 and the lower PZT element 1213 to cause desired vibrations and applies the drive signals to the piezoelectric elements. The drive signals are alternating voltages. The drive signal generator 132 sets a phase difference between two drive signals in accordance with the moving direction. The drive signal generator 132 sets an amplitude of the voltage of each drive signal or a duty ratio of the drive signals in accordance with the moving speed.

As described above, the stretching vibration is caused when the same voltage is applied to all the four divided electrodes, and the bending vibration is caused when the same voltage is applied to electrodes located diagonally and voltages having opposite polarities are applied to electrodes adjacent to each other among the four divided electrodes. When a direction of the elliptical movement of the contact section 1215, which is caused by combination of the stretching vibration and the bending vibration, is changed in response to the command input by the operator, the rotation directions of the front in-wheel motor 1114 and the rear in-wheel motor 1115 are changed.

The drive signal generator 132 changes, on the basis of the command of the operator, drive signals supplied to the four divided electrodes of each of the in-wheel motors, thereby changing the rotation direction of each of the in-wheel motors, and realizing the translation and rotation of the insertion section 201 according to the command of the operator.

### (Effect of Insertion-Section Conveying Unit 100)

With the configuration described above, in the in-wheel motors according to the present embodiment, the pressure with which the pantograph preload mechanisms 150 and 151 press the ultrasonic vibrator 12 against the housing 16 is able to be easily adjusted.

Accordingly, an in-wheel motor of an internal contact type capable of performing tuning that has little effect on vibration characteristics of an ultrasonic vibrator is able to be realized.

In the medical apparatus 1 of the present embodiment, by using a differential drive mechanism 110A which uses such an in-wheel motor, no gear is required, and a small-sized, silent, and highly reliable insertion-section conveying unit is provided.

### Embodiment 2

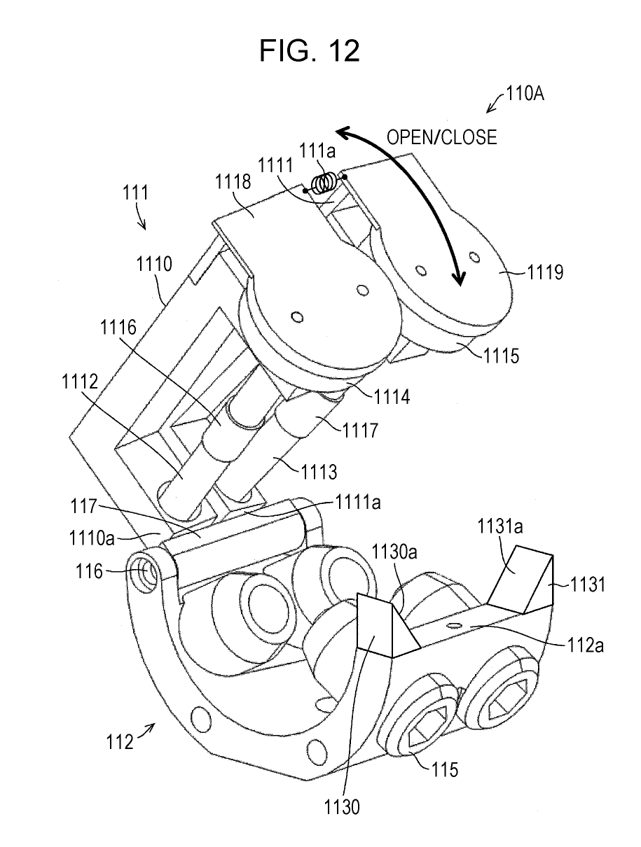

Another embodiment of the invention will be described below with reference to FIGS. 12 to 14. Note that, for convenience of description, members having the same functions as those of the members described in the aforementioned embodiment will be given the same reference signs and description thereof will be omitted.

A differential drive mechanism 110A according to the present embodiment includes an angle changing mechanism that changes crossing angles between the front conveyance roller 1112 and the insertion section 201 and between the rear conveyance roller 1113 and the insertion section 201, in addition to the configuration of the differential drive mechanism 110 described above. With such a configuration, the crossing angles θ (refer to FIG. 5) of the front conveyance roller 1112 and the rear conveyance roller 1113 relative to the insertion section 201 are able to be adjusted.

### (Entire Configuration)

FIG. 12 illustrates an overview of the differential drive mechanism 110A according to the present embodiment.

As illustrated in FIG. 12, the differential drive mechanism 110A includes the upper housing unit 111, the lower housing unit 112, the coupling section 117, and the preload spring (restoring section) 116.

The upper housing unit 111 includes the front arm 1110, the rear arm 1111, and a spring 111a. The front arm 1110 and the rear arm 1111 in the present embodiment are configured to be rotatable with contact sections 1110a and 1111a with the lower housing unit 112 as axes. The spring 111a is a spring whose one end is connected to the front arm 1110 and whose other end is connected to the rear arm 1111 and is configured to separate the front arm 1110 and the rear arm 1111 from each other.

The lower housing unit 112 includes the four ball bearings 115 and guides 1130 and 1131.

The guides 1130 and 1131 are a pair of members for guiding the front in-wheel motor 1114 and the rear in-wheel motor 1115 in a process where the upper housing unit 111 and the lower housing unit 112 are closed. The guides 1130 and 1131 are arranged on an end surface 112a of the lower housing unit 112, against which the front in-wheel motor 1114 and the rear in-wheel motor 1115 abut in a state where the upper housing unit 111 and the lower housing unit 112 are closed.

The guides 1130 and 1131 respectively have inclined surfaces 1130a and 1131a that contact with the front in-wheel motor 1114 and the rear in-wheel motor 1115 in the process where the upper housing unit 111 and the lower housing unit 112 are closed. The inclined surfaces 1130a and 1131a are placed at positions facing each other and a distance between the inclined surface 1130a and the inclined surface 1131a decreases as being closer to the end surface 112a. Thus, as a distance between the upper housing unit 111 and the lower housing unit 112 is shorter, the front in-wheel motor 1114 and the rear in-wheel motor 1115 are guided by the inclined surfaces 1130a and 1131a, so that a distance between the front in-wheel motor 1114 and the rear in-wheel motor 1115 decreases, resulting in reduction in an angle formed by the front conveyance roller 1112 and the rear conveyance roller 1113.

As described above, the angle changing mechanism in the differential drive mechanism 110A according to the present embodiment includes the spring 111a that urges the front conveyance roller 1112 and the rear conveyance roller 1113 so that a distance between one distal ends of the front conveyance roller 1112 and the rear conveyance roller 1113 increases, and the guides 1130 and 1131 that restrict the distance between the distal ends in accordance with the distances from the front conveyance roller 1112 and the rear conveyance roller 1113 to the ball bearings 115. The angle changing mechanism changes the crossing angles θ in conjunction with the distances from the front conveyance roller 1112 and the rear conveyance roller 1113 to the ball bearings 115, which are changed by the coupling section 117. The angle changing mechanism changes the crossing angles so that the crossing angle θ by the front conveyance roller 1112 and the crossing angle θ by the rear conveyance roller 1113 are the same as each other.

### (Operation of Angle Changing Mechanism)

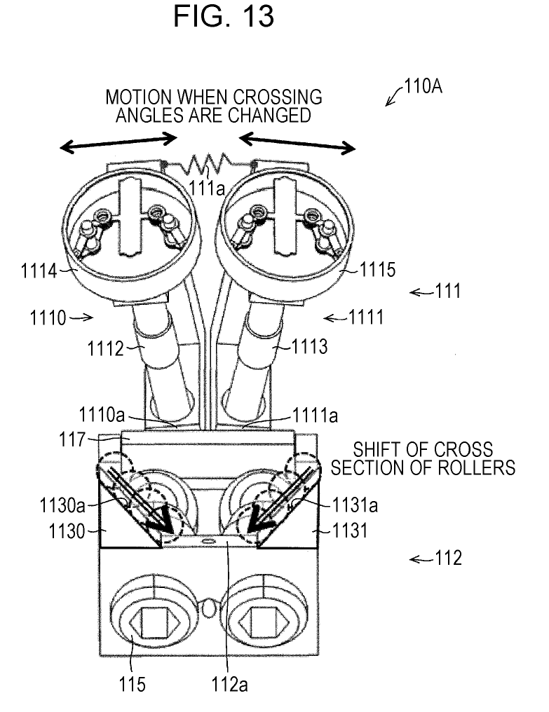

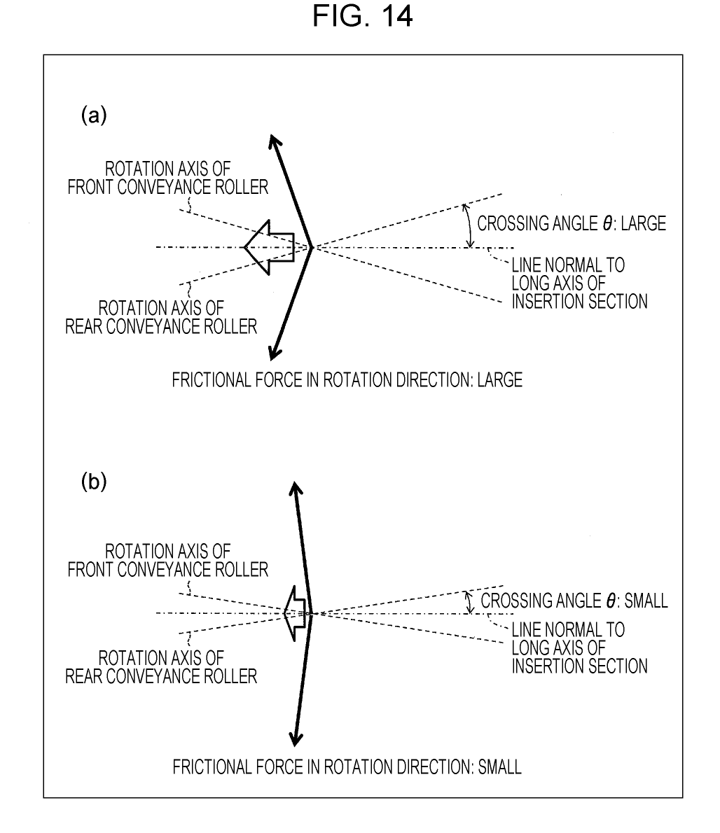

FIG. 13 illustrates positions of the front conveyance roller 1112 and the rear conveyance roller 1113 on the guides 1130 and 1131. When an outer diameter of the insertion section 201 is large, the upper housing unit 111 and the lower housing unit 112 are separated from each other. Thus, the front in-wheel motor 1114 and the rear in-wheel motor 1115 move away from the end surface 112a, so that a space where both of them are separated from each other is provided. At this time, due to an operation of the spring 111a, the front in-wheel motor 1114 and the rear in-wheel motor 1115 are urged in directions to be separated from each other and an angle formed by the front conveyance roller 1112 and the rear conveyance roller 1113 becomes large. As a result, the crossing angles θ between the line normal to the long axis of the insertion section 201 and the front conveyance roller 1112 and rear conveyance roller 1113 become large (refer to FIG. 5).

On the other hand, when the outer diameter of the insertion section 201 is small, the upper housing unit 111 and the lower housing unit 112 come close to each other. Thus, the front conveyance roller 1112 and the rear conveyance roller 1113 are respectively guided by the guides 1130 and 1131, so that the front arm 1110 and the rear arm 1111 come close to each other. At this time, the crossing angles θ become small.

Views (a) and (b) of FIG. 14 each illustrate a resultant vector, in a direction in which the insertion section 201 rotates, of frictional force generated through rotation of the front conveyance roller 1112 and the rear conveyance roller 1113.

When the crossing angles θ are large, the resultant vector, in the direction in which the insertion section 201 rotates, of the frictional force generated through rotation of the front conveyance roller 1112 and the rear conveyance roller 1113 is large as illustrated in (a) of FIG. 14. Thus, when the front conveyance roller 1112 and the rear conveyance roller 1113 rotate at a predetermined speed, a rotation speed of the insertion section 201 increases.

When the outer diameter of the insertion section 201 is large, the crossing angles θ are automatically set to be large in the differential drive mechanism 110A. Thus, when the outer diameter of the insertion section 201 is large, the rotation speed of the insertion section 201 increases.

On the other hand, when the crossing angles θ are small, the resultant vector, in the direction in which the insertion section 201 rotates, of the aforementioned frictional force is small as illustrated in (b) of FIG. 14, so that when the front conveyance roller 1112 and the rear conveyance roller 1113 rotate at the predetermined speed, the rotation speed of the insertion section 201 decreases.

When the outer diameter of the insertion section 201 is small, the crossing angles θ are automatically set to be small in the differential drive mechanism 110A. Thus, when the outer diameter of the insertion section 201 is small, the rotation speed of the insertion section 201 decreases.

A relation of the outer diameter of the insertion section 201 and the crossing angles θ is able to be appropriately set in accordance with shapes of the guides 1130 and 1131, in particular, inclination angles of the inclined surfaces relative to the end surface 112a.

With the differential drive mechanism 110A according to the present embodiment, by appropriately setting the shapes of the guides 1130 and 1131, appropriate crossing angles according to the outer diameter of the insertion section 201 are automatically set. Thereby, an appropriate rotation speed according to the outer diameter of the insertion section 201 is automatically set.

Moreover, with the differential drive mechanism 110A according to the present embodiment, any desired movement out of the translation movement and the rotation movement of the insertion section 201 is enabled to be selectively executed because the crossing angle by the front conveyance roller 1112 and the crossing angle by the rear conveyance roller 1113 are equal to each other.

Further, with the differential drive mechanism 110A according to the present embodiment, driving by an appropriate combination of a rotation speed and a torque of a motor is able to be achieved whether the driving direction is the translation direction or the rotation direction. A rotation speed and a torque of a motor generally has an inverse correlation, and a DC motor or a stepping motor in which such a correlation of a rotation speed and a torque of a motor is linear is particularly desired to be driven by a combination of a rotation speed and a torque, with which the greatest force is provided or the highest power efficiency is achieved. The driving with such a preferable combination is able to be achieved because the crossing angles θ are able to be adjusted by adjusting the distance between the guides 1130 and 1131 or the angles of the inclined surfaces 1130a and 1131a in the differential drive mechanism 110A.

Note that, when force by the spring 111a is larger than force by the preload spring 116, the front arm 1110 and the rear arm 1111 may be guided by the guides 1130 and 1131 and rise from the insertion section 201. Thus, the force by the spring 111a is preferably smaller than the force by the preload spring 116.

### Embodiment 3

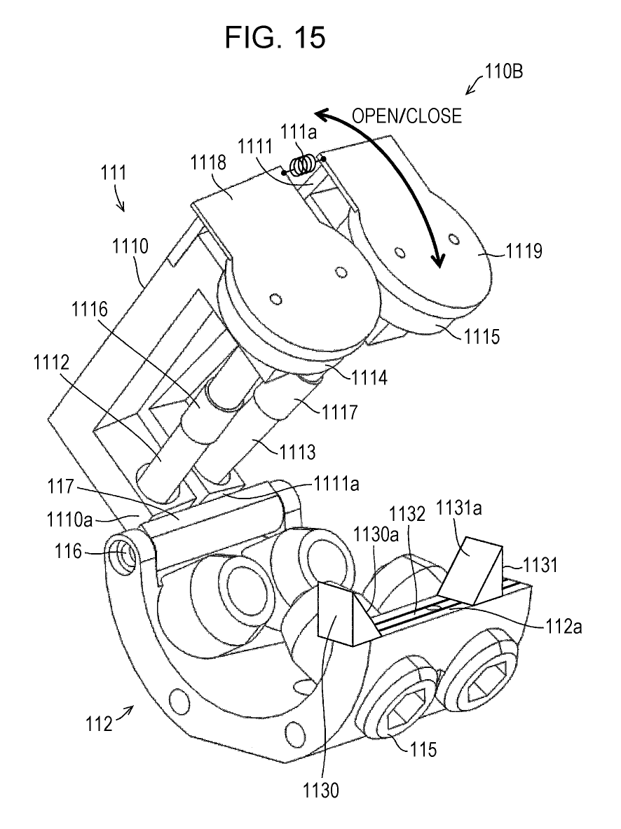

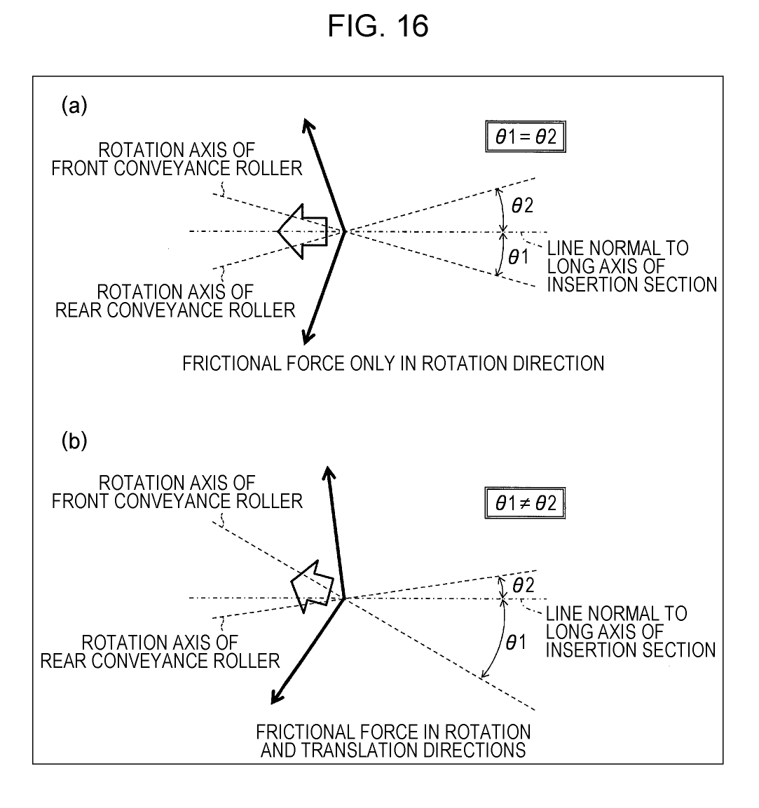

Another embodiment of the invention will be described below with reference to FIGS. 15 and 16. Note that, for convenience of description, members having the same functions as those of the members described in the aforementioned embodiments will be given the same reference signs and description thereof will be omitted.

A differential drive mechanism 110B according to the present embodiment includes a lane 1132 in addition to the configuration of the differential drive mechanism 110A described above. The guides 1130 and 1131 are configured to be movable on the lane 1132. With such a configuration, the front conveyance roller 1112 and the rear conveyance roller 1113 are able to have different crossing angles θ.

### (Entire Configuration)

FIG. 15 illustrates an overview of the differential drive mechanism 110B according to the present embodiment.

As illustrated in FIG. 15, the differential drive mechanism 110B includes the upper housing unit 111, the lower housing unit 112, the coupling unit 117, and the preload spring (restoring section) 116.

The lower housing unit 112 includes the four ball bearings (sliders) 115, the guides 1130 and 1131, and the lane 1132. The lane 1132 is a groove for changing the distance between the guides 1130 and 1131. The lane 1132 is formed on the end surface 112a and is formed so as to be parallel to the long axis of the insertion section 201 when the insertion section 201 is installed in the differential drive mechanism 110B.

The guides 1130 and 1131 and the lane 1132 constitute a main part of the aforementioned angle changing mechanism that changes the crossing angles. By moving the guides 1130 and 1131 along the lane 1132, the crossing angles are able to be changed so that the crossing angle of the front conveyance roller 1112 and the crossing angle of the rear conveyance roller 1113 are different from each other.

### (Operation of Angle Changing Mechanism)

Views (a) and (b) of FIG. 16 each illustrate a resultant vector of frictional force when the front conveyance roller 1112 and the rear conveyance roller 1113 rotate as illustrated in FIG. 5. Here, (a) of FIG. 16 illustrates a case where a crossing angle θ1 of the front conveyance roller 1112 and a crossing angle θ2 of the rear conveyance roller 1113 are the same and (b) of FIG. 16 illustrates a case where the crossing angle θ1 and the crossing angle θ2 are different from each other.

Hereinafter, when the crossing angle θ1 and the crossing angle θ2 are the same, the crossing angles are considered to be symmetrical, and when the crossing angle θ1 and the crossing angle θ2 are different from each other, the crossing angles are considered to be unsymmetrical.

In a case where the crossing angles are symmetrical, when the front conveyance roller 1112 and the rear conveyance roller 1113 rotate in directions different from each other as illustrated in FIG. 5, the resultant vector of frictional force generated between the insertion section 201 and each of the front conveyance roller 1112 and the rear conveyance roller 1113 is, as illustrated in (a) of FIG. 16, in a direction vertical to the long axis of the insertion section 201, that is, a direction in which the insertion section 201 rotates, so that the insertion section 201 rotates about the long axis.

On the other hand, when the front conveyance roller 1112 and the rear conveyance roller 1113 rotate in the same direction with each other as illustrated in FIG. 4, the resultant vector is in a direction parallel to the long axis of the insertion section 201, that is, a direction in which the insertion section 201 is translated, so that the insertion section 201 is translated in the direction parallel to the long axis.

That is, when the crossing angles are symmetrical, the insertion section 201 performs only either the rotation movement or the translation movement in accordance with the rotation directions of the front conveyance roller 1112 and the rear conveyance roller 1113.

On the other hand, in the differential drive mechanism 110B according to the present embodiment, the crossing angles are able to be unsymmetrical by individually moving the guides 1130 and 1131 along the lane 1132 as described above.

When the front conveyance roller 1112 and the rear conveyance roller 1113 rotate in directions different from each other as illustrated in FIG. 5 in a state where the crossing angles are unsymmetrical, the resultant vector of frictional force generated between the insertion section 201 and each of the front conveyance roller 1112 and the rear conveyance roller 1113 is in a direction oblique to both the long axis of the insertion section 201 and the line normal to the long axis of the insertion section 201 as illustrated in (b) of FIG. 16. That is, the resultant vector has both a vertical component and a horizontal component with respect to the long axis. In this case, the insertion section 201 performs both the rotation and translation movement at the same time.

Thus, when the insertion section 201 is inserted while being rotated at a fixed speed, the positions of the guides 1130 and 1131 may be decided in advance so that a desired ratio of the rotation speed and the translation speed is obtained. As a result, when inserting the insertion section 201, the operator is able to easily perform the insertion operation of the insertion section 201 without instructing or adjusting both the rotation movement and translation movement. An example of such an insertion section 201 includes a thrombus removal catheter.

### Embodiment 4

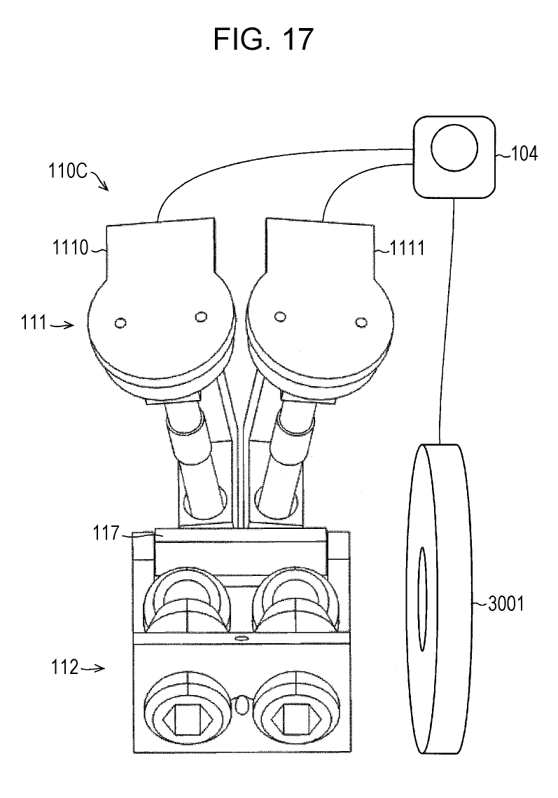

Another embodiment of the invention will be described below with reference to FIG. 17. Note that, for convenience of description, members having the same functions as those of the members described in the aforementioned embodiments will be given the same reference signs and description thereof will be omitted.

FIG. 17 illustrates a differential drive mechanism 110C according to the present embodiment. As illustrated in FIG. 17, the differential drive mechanism 110C according to the present embodiment includes an insertion section movement detection sensor 3001 that detects a speed at which the insertion section 201 is translated or rotates, in addition to the configuration of the differential drive mechanism 110. With such a configuration, when there is a difference of a frictional coefficient between each of the conveyance rollers and the insertion section 201 and the translation speed and the rotation speed of the insertion section 201 are different from assumed speeds, the rotation speeds of the conveyance rollers are able to be corrected to suppress unintended movement of the insertion section 201.

Specifically, the insertion section movement detection sensor 3001 uses optical movement detection means whose technique has been established, for example, for an optical mouse for controlling a personal computer, or a non-contact measurement method such as a magnetic detection method.

When the optical movement detection means is used, the insertion section movement detection sensor 3001 includes, for example, an image sensor and acquires images on a surface of the insertion section 201 at a sufficiently short and predetermined cycle. From regions matched in continuous images, the insertion section movement detection sensor 3001 reads a movement amount of the insertion section 201 between the images and calculates a movement speed of the insertion section 201 on the basis of the movement amount and the cycle.

In general, on the assumption that frictional coefficients of two conveyance rollers are the same, rotation speeds of the conveyance rollers are set in a differential drive mechanism. However, it is considered that there is a difference between the frictional coefficients of the two conveyance rollers when contamination such as blood adheres to an insertion section.

Thus, a control unit 104 of the differential drive mechanism 110C according to the present embodiment monitors the translation speed and the rotation speed of the insertion section 201 by the insertion section movement detection sensor 3001, and, when scheduled movement is different from actual movement of the insertion section 201, corrects the rotation speeds of the rollers. Accordingly, a safer treatment is able to be performed.

For example, in a case where unintended translation movement of the insertion section 201 directed upward in FIG. 5 is detected when the insertion section 201 is moved to rotate in a direction of an arrow according to arrangement in FIG. 5, the speed at which the rear conveyance roller 1113 conveys the insertion section 201 is considered to be higher than the speed at which the front conveyance roller 1112 conveys the insertion section 201. Thus, by decreasing the rotation speed of the rear conveyance roller 1113 or increasing the rotation speed of the front conveyance roller 1112, the unintended translation movement is able to be suppressed.

### Embodiment 5

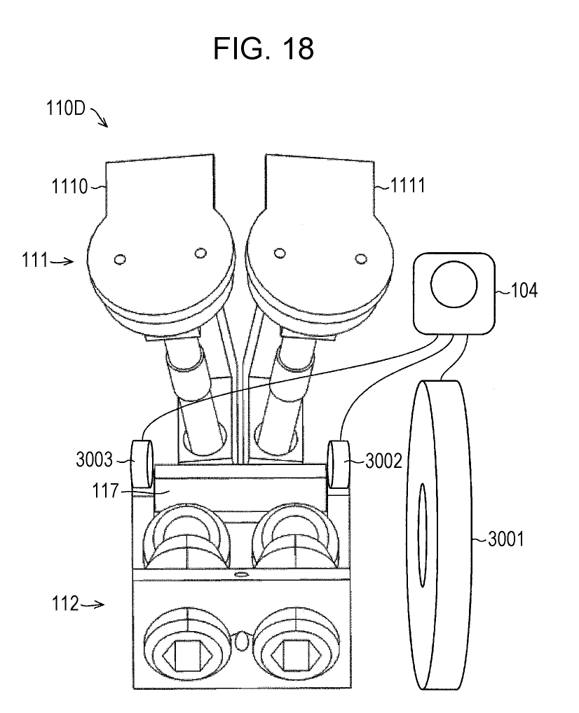

Another embodiment of the invention will be described below with reference to FIG. 18. Note that, for convenience of description, members having the same functions as those of the members described in the aforementioned embodiments will be given the same reference signs and description thereof will be omitted.

FIG. 18 illustrates a differential drive mechanism 110D according to the present embodiment. As illustrated in FIG. 18, the differential drive mechanism 110D according to the present embodiment includes pressing force adjustment mechanisms 3002 and 3003 that adjust force by which the front arm 1110 and the rear arm 1111 are pressed against the insertion section 201, in addition to the configuration of the differential drive mechanism 110C. With such a configuration, when there is a difference of a frictional coefficient between the conveyance rollers and the insertion section 201 and the translation speed and the rotation speed of the insertion section 201 are different from assumed speeds, the pressing force of the conveyance rollers against the insertion section 201 is able to be corrected to suppress the unintended movement of the insertion section 201.

In general, on the assumption that frictional coefficients of two conveyance rollers are the same, rotation speeds of the conveyance rollers are set in a differential drive mechanism. However, it is considered that there is a difference between the frictional coefficients of the two conveyance rollers when contaminant such as blood adheres to an insertion section.

Thus, the control unit 104 of the differential drive mechanism 110D according to the present embodiment monitors the translation speed and the rotation speed of the insertion section 201 by the insertion section movement detection sensor 3001, and when scheduled movement is different from actual movement of the insertion section 201, corrects the pressing force of the front arm 1110 and the rear arm 1111 by the pressing force adjustment mechanisms 3002 and 3003, thus making it possible to perform a safer treatment.

The pressing force adjustment mechanisms 3002 and 3003 are control mechanisms that control the pressing force of the front conveyance roller 1112 and the rear conveyance roller 1113 against the insertion section 201. Specifically, the pressing force adjustment mechanisms 3002 and 3003 each include a spiral spring and a motor. The spiral spring has a center end connected to the motor and has a peripheral end connected to the front arm 1110 or the rear arm 1111.

When the motor rotates, in accordance with a direction of the rotation, force for closing or opening the front arm 1110 or the rear arm 1111 and the lower housing unit 112 is generated, so that force by which the front conveyance roller 1112 or the rear conveyance roller 1113 is pressed against the insertion section 201 changes.

Thus, when the motor of the pressing force adjustment mechanism 3002 or 3003 rotates, the force by which the front conveyance roller 1112 or the rear conveyance roller 1113 is pressed against the insertion section 201 is able to be adjusted.

A signal which is output from the insertion section movement detection sensor 3001 and relates to the translation speed and the rotation speed of the insertion section 201 is fed back to the control unit 104, and the pressing force is corrected by the pressing force adjustment mechanisms 3002 and 3003 provided in the front arm 1110 and the rear arm 1111.

For example, according to the arrangement in FIG. 5, in a case where unintended translation movement of the insertion section 201 directed upward in FIG. 5 is detected when the insertion section 201 is moved to rotate in the direction of the arrow, the speed at which the rear conveyance roller 1113 conveys the insertion section 201 is considered to be higher than the speed at which the front conveyance roller 1112 conveys the insertion section 201. Thus, by decreasing the pressing force of the rear conveyance roller 1113 or increasing the pressing force of the front conveyance roller 1112, the unintended translation movement is able to be suppressed.

### CONCLUSION

An ultrasonic actuator according to an aspect 1 of the invention includes: an ultrasonic vibrator (12); a rotor (housing 16) that rotates with vibration of the ultrasonic vibrator; and preload mechanisms (pantograph preload mechanisms 150 and 151) that hold the ultrasonic vibrator at a node of vibration of the ultrasonic vibrator and generate pressure with which the ultrasonic vibrator is pressed against the rotor.

According to the aforementioned configuration, the ultrasonic actuator includes the ultrasonic vibrator, the rotor, and the preload mechanisms. The rotor rotates with vibration of the ultrasonic vibrator. The preload mechanisms hold the ultrasonic vibrator at the node of vibration of the ultrasonic vibrator and generate pressure with which the ultrasonic vibrator is pressed against the rotor.

Since the preload mechanisms hold the ultrasonic vibrator at the node of vibration of the ultrasonic vibrator, little effect is applied to vibration characteristics of the ultrasonic vibrator even when the pressure with which the ultrasonic vibrator is pressed against the rotor changes.

Thus, it is possible to provide an ultrasonic actuator on which tuning that has little effect on vibration characteristics of an ultrasonic vibrator is able to be performed.

The ultrasonic actuator according to an aspect 2 of the invention may be configured, in the aspect 1, so that each of the preload mechanisms includes a slide support section (guide roller 1516) that contacts with an inner surface of the rotor, the rotor is held from inside the rotor by a distal end (contact section 1215) of the ultrasonic vibrator and the slide support section, and the preload mechanism generates force in a direction in which a distance between the node of the vibration and the slide support section increases.

According to the aforementioned configuration, each of the preload mechanisms includes the slide support section that contacts with the inner surface of the rotor. The rotor is held from inside the rotor by the distal end of the ultrasonic vibrator and the slide support section. The preload mechanism generates force in the direction in which the distance between the node of the vibration of the ultrasonic vibrator and the slide support section increases, thereby adjusting the pressure with which the ultrasonic vibrator is pressed against the rotor.

Thus, the distal end of the ultrasonic vibrator and the slide support section make it possible to suitably perform holding of the rotor and adjustment of the pressure with which the ultrasonic vibrator is pressed against the rotor.

The ultrasonic actuator according to an aspect 3 of the invention may be configured, in the aspect 1 or 2, so that the ultrasonic vibrator has holders (1214) each of which is a protrusion formed at the node of the vibration, and the preload mechanisms are connected to the holders.

According to the aforementioned configuration, the preload mechanisms are connected to the holders each of which is the protrusion formed at the node of the vibration of the ultrasonic vibrator.

Thus, it is possible to further reduce the effect on the vibration of the ultrasonic vibrator due to holding of the ultrasonic vibrator by the preload mechanisms.

The ultrasonic actuator according to an aspect 4 of the invention may be configured, in the aspect 3, so that the holders are formed to be positioned symmetrical about a long axis of the ultrasonic vibrator, and each of the preload mechanisms is connected to a corresponding one of a pair of holders.

According to the aforementioned configuration, each of the preload mechanisms is connected to the corresponding one of the pair of holders formed to be positioned symmetrical about the long axis of the ultrasonic vibrator.

Thus, it is possible to stably hold the ultrasonic vibrator and also to adjust a contact angle, at which the ultrasonic vibrator contacts with the rotor, by differentiating the pressure generated by each of the preload mechanisms.

The ultrasonic actuator according to an aspect 5 of the invention may be configured, in any of the aspects 1 to 4, so that each of the preload mechanisms includes a pair of arms, one end of the pair of arms is connected to the ultrasonic vibrator with an angle formed, and the preload mechanism adjusts the pressure by adjusting the angle formed by the one end.

According to the aforementioned configuration, the one end of the pair of arms provided in the preload mechanism is connected to the ultrasonic vibrator with the angle formed. By adjusting the angle, the preload mechanism adjusts the pressure with which the ultrasonic vibrator is pressed against the rotor.

Thus, it is possible to realize a preload mechanism with a simple structure.

The ultrasonic actuator according to an aspect 6 of the invention may further include, in the aspect 5, an adjustment member (adjustment screw 1514) that adjusts the angle, in which a part of the adjustment member may be exposed to an outer surface of a housing of the ultrasonic actuator.

According to the aforementioned configuration, the ultrasonic actuator includes the adjustment member that adjusts the angle formed by the end of the pair of arms. A part of the adjustment member is exposed to the outer surface of the housing of the ultrasonic actuator.

Thus, since the angle is able to be adjusted without opening the housing of the ultrasonic actuator, it is possible to easily adjust the pressure, with which the ultrasonic vibrator is pressed against the rotor, after the ultrasonic actuator is manufactured.

The ultrasonic actuator according to an aspect 7 of the invention may be configured, in any of the aspects 1 to 6, so that the rotor is provided with a guide groove that restricts a position where the ultrasonic vibrator contacts with the rotor.

According to the aforementioned configuration, the rotor is provided with the guide groove that restricts the position where the ultrasonic vibrator contacts with the rotor.

Thus, the ultrasonic vibrator is able to rotate the rotor stably.

The invention is not limited to the embodiments described above and may be modified in various manners within the scope of the claims, and an embodiment achieved by appropriately combining technical means disclosed in different embodiments is also encompassed in the technical scope of the invention. Further, by combining the technical means disclosed in each of the embodiments, a new technical feature may be formed.

### INDUSTRIAL APPLICABILITY

The invention is able to be used as a small-sized motor and is able to be suitably used, in particular, in a medical apparatus or a small-sized robot.

### REFERENCE SIGNS LIST

100 insertion-section conveying unit
130 control unit
111 upper housing unit
112 lower housing unit
1114 front in-wheel motor (ultrasonic actuator)
1115 rear in-wheel motor (ultrasonic actuator)
12 ultrasonic vibrator
1214 holder
150, 151 pantograph preload mechanism
1501a, 1501b, 1502a, 1502b arm
1514 adjustment screw (adjustment member)
1516 guide roller (slide support section)
16 housing (rotor)
1605 guide groove
201 insertion section

1. An ultrasonic actuator comprising
a rotor in a ring shape; and
an ultrasonic vibrator internally contacts with the rotor, wherein
the ultrasonic vibrator is held by a plurality of preload mechanisms, and
each of the preload mechanisms is connected to the ultrasonic vibrator near a node of vibration of the ultrasonic vibrator and has a slide support section that contacts with an inner surface of the rotor at an end that is not connected to the ultrasonic vibrator, and
the rotor is held from inside the rotor by a distal end of the ultrasonic vibrator and the slide support section.

2. The ultrasonic actuator according to claim 1, wherein
the preload mechanisms generate force in a direction in which a distance between the node of the vibration and the slide support section increases.

3. The ultrasonic actuator according to claim 1, wherein
the ultrasonic vibrator has holders each of which is a protrusion formed at the node of the vibration, and
the preload mechanisms are connected to the holders.

4. The ultrasonic actuator according to claim 3, wherein
the holders are formed to be positioned symmetrically about a long axis of the ultrasonic vibrator, and
each of the preload mechanisms is connected to a corresponding one of a pair of the holders.

5. The ultrasonic actuator according to claim 1, wherein
each of the preload mechanisms includes a pair of arms,
one end of the pair of arms is connected to the ultrasonic vibrator with an angle formed, and
the preload mechanism adjusts the pressure, with which the ultrasonic vibrator is pressed against the rotor, by adjusting the angle formed by the one end.

6. The ultrasonic actuator according to claim 5, further comprising
an adjustment member that adjusts the angle, wherein
a part of the adjustment member is exposed to an outer surface of a housing of the ultrasonic actuator.

7. The ultrasonic actuator according to claim 1, wherein
the rotor is provided with a guide groove that restricts a position where the ultrasonic vibrator contacts with the rotor.
